(19) United States
(12) Patent Application Publication
(10) Pub. No.: US 2011/0136851 A1
(43) Pub. Date: Jun. 9, 2011

(54) Sucralose Formulations To Mask Unpleasant Tastes

(75) Inventors: David Jaeger, Chester, VA (US); Jay Dickerson, Midlothian, VA (US); Annabelle Trimmer, Manakin Sabot, VA (US)

(73) Assignee: Wyeth LLC, Madison, NJ (US)

(21) Appl. No.: 13/018,932

(22) Filed: Feb. 1, 2011

Related U.S. Application Data
(63) Continuation of application No. 10/485,858, filed on Jan. 27, 2005, now abandoned.
(60) Provisional application No. 60/308,912, filed on Jul. 31, 2001.

(51) Int. Cl. A61K 31/485; A61K 31/4402; A61K 31/09; A61K 31/137; A61K 31/192; A61K 31/167; A61P 29/00; A61P 11/14; A61P 11/10; A61P 11/02

(52) U.S. Cl. 514/289; 514/357; 514/718; 514/653; 514/570; 514/569; 514/629

(57) ABSTRACT

The present invention is directed to a pharmaceutically acceptable taste masking liquid excipient base for administration of a relatively large amount of unpleasant tasting medicines. More particularly, the enhanced sweetness and taste masking effect are produced by the addition of sucralose to the excipient base with maintenance of a pH from about 2 to about 5. The invention is further directed to medicinal compositions comprising such a liquid excipient base and unpleasant tasting medicines. Still further, the invention is directed to a method for taste masking unpleasant tasting medicines through their incorporation into the claimed liquid excipient bases.

This application claims priority under 35 U.S.C. §1.119(e) to provisional application Ser. No. 60/308,912, filed Jul. 31, 2001, which is incorporated herein by reference in its entirety.

### FIELD OF THE INVENTION

The present invention is directed to a pharmaceutically acceptable taste masking liquid excipient base for administration of a relatively large amount of unpleasant tasting medicines. More particularly, the enhanced sweetness and taste masking effect are produced by the addition of sucralose to the excipient base with maintenance of a pH from about 2 to about 5. The invention is further directed to medicinal compositions comprising such a liquid excipient base and unpleasant tasting medicines. Still further, the invention is directed to a method for taste masking unpleasant tasting medicines through their incorporation into the claimed liquid excipient bases.

### BACKGROUND OF THE INVENTION

Pharmaceutically acceptable liquid excipient bases for administration of unpleasant tasting medicines are well known in the art. A typical system is described in U.S. Pat. No. 5,260,073 to Roger J. Phipps at column 7 as including a medicine, a solvent, a co-solvent, a buffer, a surfactant, a preservative, a sweetening agent, a flavoring agent, a dye or pigment, a viscosity modifier and water. The patent provides several examples of each ingredient in the system. However, despite making the medicine more palatable, these compositions retain an unpleasant taste and have room for improvement.

Although liquid excipient bases and their many ingredients are well known, unpleasant tasting medicines alone or in combination still present challenges to one skilled in the art to provide better taste masked products and, in certain instances, to provide taste masking for higher dosage amounts of unpleasant tasting medicines in smaller amounts of vehicle.

### SUMMARY OF THE INVENTION

The present invention provides a pharmaceutically acceptable taste masking liquid excipient base for administration of unpleasant tasting pharmaceutically active compounds. The liquid base contains a sufficient amount of sucralose to mask a bitter taste of any active ingredients dissolved therein and has a pH of less than about 5.0 and greater than about 2.0. Lower pH and use of sucralose as a sweetener achieves an unexpected synergy of bitter taste masking effectiveness.

Pharmaceutically acceptable taste masking bases useful in this invention are preferably those incorporating therein, per 100 milliliters of base, about 50 to about 300 milligrams, preferably about 100 to about 200 milligrams, most preferably 200 milligrams of sucralose. More or less sucralose may be used to achieve an acceptable balance of sweetness and effectiveness in bitter-masking. Preferably, the pH of the liquid base is from about 2 to about 5.

The excipient base may be used to mask the bitterness of any active ingredient, including those selected from the group consisting of antihistamines, decongestants, antitussives, expectorants, antibiotics, antineoplastics, non-steroidal anti-inflammatory drugs (NSAIDs) and analgesic drugs.

The invention further provides a method for taste masking at least one unpleasant tasting pharmaceutically active compound comprising dissolving such unpleasant tasting pharmaceutically active compound into a liquid excipient base containing a sufficient amount of sucralose to mask a bitter taste of any active ingredient, wherein the composition has a pH greater than 2.0 and less than 5.0.

### DETAILED DESCRIPTION OF THE INVENTION

The present invention advantageously provides an enhancement of flavor masking with sucralose. In particular, by providing a sweetening amount of sucralose, i.e., an amount of sucralose sufficient to mask the bitter taste, at an acid pH (i.e., pH less than about 5.0) in an aqueous base excipient, the invention greatly masks the bad taste, particularly bitter taste, of many actives.

The present invention is based, in part, on the discovery that modulation of pH combined with the use of sucralose as a sweetening agent produces surprisingly superior taste masking abilities—better than either manipulation produces alone and better than the combination of the two would be expected to produce.

Sucralose is a chlorinated sucrose derivative, in which three —OH groups are replaced by chlorine atoms. Sucralose has a sweet, sugar-like taste. It is approximately 600 times sweeter than sugar and is used as an artificial sweetener. Sucralose displays a rapid onset of sweetness and maintains a similar sweetness duration to sugar.

Pharmaceutically acceptable taste masking bases useful in this invention are preferably those incorporating therein per 100 milliliters of base, about 50 to about 300 milligrams, preferably about 100 to about 200 milligrams, most preferably 200 milligrams of sucralose. More or less sucralose may be used to achieve an acceptable balance of sweetness and effectiveness in bitter-masking. The pH of the liquid base is from about 2.0 to about 5.0. The preferred pH range for formulations containing acetaminophen combined with pseudoephedrine hydrochloride, dextromethorphan hydrobromide, chlorpheniramine maleate and brompheniramine maleate is 4.0-5.0. The preferred pH range for formulations containing guaifenesin combined with pseudoephedrine hydrochloride and/or dextromethorphan hydrobromide is 2.0-4.0. In a specific embodiment the lower value is about 2.5 or 2.6; in another embodiment the upper value is about 3.7.

In accordance with the invention, pH of the base can be lowered by addition of acids, e.g., citric acid, or any other pharmaceutically acceptable acid. Alternatively, pH is established using a buffer system. Preferably the buffer is sodium citrate/citric acid. However, any pharmaceutically acceptable buffer can be used, including phosphate, acetate, maleate, and any other acid or acid-salt.

Citrate buffering systems comprising, per 100 milliliters of base, (i) about 200 to about 1800, preferably about 300 to about 1700 milligrams, most preferably 400 or 1600 milligrams of citric acid; and (ii) about 0 to about 200 milligrams, preferably about 50 to about 150 milligrams, most preferably about 100 milligrams of sodium citrate are used to yield a pH in the range of about 2 to about 5. Preferably, the pH is established after the addition of active ingredients and sucralose.

The pharmaceutically active compounds useful in the practice of the present invention include antihistamines, decongestants, antitussives, expectorants, antibiotics, antineoplastics, non-steroidal anti-inflammatory drugs (NSAIDs) and other analgesic drugs such as acetominophen and phenacetin. These materials are incorporated into the claimed liquid excipient base in amounts governed by the solubility of the material in such excipient base and such that conventional dosages thereof shall be in compliance with applicable FDA regulations. For example, materials highly soluble in the liquid excipient base must not be incorporated to the extent that a typical dose (such as one teaspoon) contains more of such material than permitted by such regulations.

The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “about” or “approximately” means within an acceptable error range for the particular value, which will depend on how the value is measured or determined. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

### Active Ingredients

As used herein, the term “active ingredient” or “active agent”with respect to a pharmaceutical or therapeutic product refers to one or more compounds that have some pharmacological property, Accordingly, more than one type of active ingredient compound may be added to the formulation of the invention. The formulation of the invention may comprise any active ingredient which may be orally administered to a subject. Formulations including active ingredients in amounts appropriate for the desired pharmacological properties at the dosage administration can be prepared. Placebo formulations, which lack an “active ingredient” having a known pharmacologic activity, are also within the scope of the invention. An “active ingredient” of a placebo can be any non-active compound that takes the place of an active ingredient in the corresponding formulation.

The invention is best suited for formulations in which the active is more stable at a pH less than 5.0.

A non-limiting list of acceptable active ingredients may include but is by no means limited to the following (where specific salts or esters are mentioned, it should be understood to include other salt, ester, or free acid forms of the drug):

1) antipyretic analgesic anti-inflammatory agents (which are discussed in greater detail, with additional examples, below), including non-steroidal anti-inflammatory drugs (NSAIDs) such as indomethacin, aspirin, diclofenac sodium, ketoprofen, ibuprofen, mefenamic acid, azulene, phenacetin, isopropylantipyrin, acetaminophen, benzydamine hydrochloride, phenylbutazone, flufenamic acid, mefenamic acid, sodium salicylate, choline salicylate, sasapyrine, clofezone or etodolac; and steroidal drugs such as dexamethasone, dexamethasone sodium sulfate, hydrocortisone, prednisolone;

2) antiulcer agents such as ranitidine, sulpiride, cetraxate hydrochloride, gefarnate, irsogladine maleate, cimetidine, lanitidine hydrochloride, famotidine, nizatidine or roxatidine acetate hydrochloride;

3) coronary vasodilators such as Nifedipine, isosorbide dinitrate, diltiazem hydrochloride, trapidil, dipyridamole, dilazep dihydrochloride, methyl 2,6-dimethyl-4-(2-nitrophenyl)-5-(2-oxo-1,3,2-dioxaphosphorinan-2-yl)-1,4-dihydropyridine-3-carboxylate, verapamil, nicardipine, nicardipine hydrochloride or verapamil hydrochloride;

4) peripheral vasodialtors such as ifenprodil tartrate, cinepazide maleate, cyclandelate, cinnarizine or pentoxyfyline;

5) oral antibacterial and antifungal agents such as penicillin, ampicillin, amoxicillin, cefalexin, erythromycin ethylsuccinate, bacampicillin hydrochloride, minocycline hydrochloride, chloramphenicol, tetracycline, erythromycin, fluconazole, itraconazole, ketoconazole, miconazole or terbinafine;

6) synthetic antibacterial agents such as nlidixic acid, piromidic acid, pipemidic acid trihydrate, enoxacin, cinoxacin, ofloxacin, norfloxacin, ciprofloxacin hydrochloride, or sulfamethoxazole trimethoprim;

7) antispasmodics such as popantheline bromide, atropine sulfate, oxapium bromide, timepidium bromide, butylscopolamine bromide, rospium chloride, butropium bromide, N-methylscopolamine methylsulfate, or methyloctatropine bromidebutropium bromide;

8) antitussive, anti-asthmatic agents such as theophylline, aminophylline, methylephedrine hydrochloride, procaterol hydrochloride, trimetoquinol hydrochloride, codeine phosphate, caramiphen (edisylate), sodium cromoglicate, tranilast, dextromethorphane hydrobromide, dimemorfan phosphate, clobutinol hydrochloride, fominoben hydrochloride, benproperine phosphate, tipepidine hibenzate, eprazinone hydrochloride, clofedanol hydrochloride, ephedrine hydrochloride, noscapine, calbetapentane citrate, oxeladin tannate, or isoaminile citrate;

9) broncyodilators such as diprophylline, salbutamol sulfate, clorprenaline hydrochloride, formoterol fumarate, orciprenalin sulfate, pirbuterol hydrochloride, hexoprenaline sulfate, bitolterol mesylate, clenbuterol hydrochloride, terbutaline sulfate, mabuterol hydrochloride, fenoterol hydrobromide, or methoxyphenamine hydrochloride;

10) diuretics such as furosemide, acetazolarmide, trichlormethiazide, methyclothiazide, hydrochlorothiazide, hydroflumethiazide, ethiazide, cyclopenthiazide, spironolactone, triamterene, fluorothiazide, piretanide, metruside, ethacrynic acid, azosemide, or clofenamide;

11) muscle relaxants such as chlorphenesin carbamate, tolperisone hydrochloride, eperisone hydrochloride, tizanidine hydrochloride, mephenesin, chlorozoxazone, phenprobamate, methocarbamol, chlormezanone, pridinol mesylate, afloqualone, baclofen, or dantrolene sodium;

12) brain metabolism altering drugs such as meclofenoxate hydrochloride;

13) minor tranquilizers such as oxazolam, diazepam, clotiazepam, medazepam, temazepam, fludiazepam, meprobamate, nitrazepam, or chlordiazepoxide;

14) major tranquilizers such as Sulpirid, clocapramine hydrochloride, zotepine, chlorpromazinon, or haloperidol;

15) β-blockers such as pindolol, propranolol hydrochloride, carteolol hydrochloride, metoprolol tartrate, labetalol hydrochloride, acebutolol hydrochloride, butetolol hydrochloride, alprenolol hydrochloride, arotinolol hydrochloride, oxprenolol hydrochloride, nadolol, bucumolol hydrochloride, indenolol hydrochloride, timolol maleate, befunolol hydrochloride, or bupranolol hydrochloride;

16) antiarrhythmic agents such as procainamide hydrochloride, disopyramide, ajimaline, quinidine sulfate, aprindine hydrochloride, propafenone hydrochloride, or mexiletine hydrochloride;

17) gout suppressants allopurinol, probenecid, colchicine, sulfinpyrazone, benzbromarone, or bucolome;

18) anticoagulants such as ticlopidine hydrochloride, dicumarol, or warfarin potassium;

19) antiepileptic agents such as phenytoin, sodium valproate, metharbital, or carbamazepine;

20) antihistaminics (antihistamines) such as chlorpheniramine (maleate), brompheniramine (maleate), dexchlorpheniramine (maleate), dexbrompheniramine (maleate), triprolidine (HCl), diphenhydramine (HCl), doxylamine (succinate), tripelennamine (HCl), cyproheptatine (HCl), bromodiphenhydramine (HCl), phenindamine (tartrate), pyrilamine (maleate, tannante), azatadine (maleate), cremastin fumarate, mequitazine, alimemazine tartrate, or cycloheptazine hydrochloride;

21) antiemetics such as Difenidol hydrochloride, metoclopramide, domperidone, betahistine mesylate, or trimebutine maleate;

22) hypotensives such as dimethylaminoethyl reserpilinate dihydrochloride, rescinnamine, methyldopa, prazosin hydrochloride, bunazosin hydrochloride, clonidine hydrochloride, budralazine, or urapidin;

23) sympathomimetic agents such as dihydroergotamine mesylate, isoproterenol hydrochloride, or etilefrine hydrochloride;

24) expectorants such as bromhexine hydrochloride, carbocysteine, ethyl cysteine hydrochloride, methyl cysteine hydrochloride, terpin hydrate, guaifenesin (glyceryl guaiacolate), potassium (iodide, citrate) or potassium guaicolsulfonate;

25) oral antidiabetic agents such as glibenclamide, tolbutamide, or glymidine sodium;

26) circulatory agents such as ubidecarenone or ATP-2Na;

27) iron preparations such as ferrous sulfate or dried ferrous sulfate;

28) vitamins such as vitamin B1, vitamin B2, vitamin B6, vitamin B12, vitamin C, vitamin A, vitamin D, vitamin E, vitamin K or folic acid;

29) pollakiuria remedies such as flavoxate hydrochloride, oxybutynin hydrochloride, terodiline hydrochloride, or 4-diethylamino-1,1-dimethyl-2-butynyl (I)-α-cyclohexyl-α-phenylglycolate hydrochloride monohydrate;

30) angiotensin-converting enzyme inhibitors such as enalapril maleate, alacepril, or delapril hydrochloride;

31) anti-viral agents such as trisodium phosphonoformate, didanosine, dideoxycytidine, azido-deoxythymidine, didehydro-deoxythymidine, adefovir dipivoxil, abacavir, amprenavir, delavirdine, efavirenz, indinavir, lamivudine, nelfinavir, nevirapine, ritonavir, saquinavir or stavudine; 32) high potency analgesics such as codeine, dihydrocodeine, hydrocodone, morphine, dilandid, demoral, fentanyl, pentazocine, oxycodone, pentazocine or propoxyphene; and

33) nasal decongestants such as pseudoephedrine (HCl), phenylpropanolamine (HCl) and phenylephrine (bitartrate, tannate, HBr, HCl). Note that active ingredients in the foregoing list may also have beneficial pharmaceutical effects in addition to the one mentioned.

Non-steroidal anti-inflammatory drugs (NSAIDs) for use in the practice of the present invention may be selected from any of the following categories: (1) propionic acid derivatives; (2) acetic acid derivatives; (3) fenamic acid derivatives; (4) biphenylcarboxylic acid derivatives; and (5) oxicams.

Of the propionic acid derivatives for use herein, ibuprofen, naproxen, ketoprofen, flurbiprofen, fenoprofen, suprofen, fenbufen, and fluprofen may be mentioned as preferred compounds.

Of the acetic acid derivatives for use herein, tolmetin sodium, zomepirac, sulindac and indomethacin are included.

Of the fenamic acid derivatives for use herein, mefenamic acid and meclofenamate sodium are included.

Diflunisal and flufenisal are biphenylcarboxylic acid derivatives.

The oxicams include piroxicam, sudoxicam and isoxicam.

Of course, it will be appreciated by those skilled in the art, that any of the foregoing compounds may be utilized in the form of their pharmaceutically acceptable salt forms, e.g. COO⁻Na⁺, COO⁻K⁺, or pharmaceutically acceptable ester forms, in acid form, and the like.

### Formulations

Various combinations of excipients and active agents can be used in the practice of the invention, in addition to sucralose at the effective pH.

The selection of appropriate excipients will depend, in part, on the physical qualities desired for the formulation (viscosity, tongue feel, etc.), and the nature of the active it contains.

Exemplary formulations are shown in Table 1:

### General Formulas and Ranges

General Formulas and Ranges

| | | Preferred | Most |
|---|---|---|---|
| Ingredients | Broad Range | Range | Preferred |
| Active Ingredients* | | | |
| Acetaminophen | 1.00%-5.00% | 2.00%-4.00% | 3.20% |
| Guaifenesin | 0.50%-5.00% | 1.00%-3.00% | 2.00% |
| Pseudoephedrine | 0.10-0.60% | 0.30-.040% | 0.30% |
| Hydrochloride | | | |
| Dextromethorphan | 0.01-0.50% | 0.10-0.30% | 0.10% |
| Hydrobromide | | | |
| Brompheniramine Maleate | 0.01-0.05% | 0.02-0.04% | 0.02% |
| Chlorpheniramine Maleate | 0.01-0.05% | 0.02-0.04% | 0.02% |
| Inactive Ingredients | | | |
| Sucralose | 0.05-0.30% | 0.10-0.20% | 0.20% |
| Citric Acid Anhydrous | 0.20-1.80% | 0.30-1.70% | 0.40% or |
| | | | 1.60% |
| Sodium Citrate | 0.00%-0.20% | 0.05%-0.15% | 0.10% |
| Polyethylene Glycol 1450 | 10-30% | 15-25% | 20.00% |
| Glycerine | 5-15% | 5.5-10% | 6.00% |
| Sorbitol, 70% | 2.00%-20.0% | 4.00%-10.0% | 5.00% |
| Sodium Benzoate | 0.10%-0.50% | 0.20%-0.40% | 0.30% |
| High Fructose Corn | 15-55% | 25-50% | 45.00% |
| Syrup 55% | | | |
| Colorant | | | |
| Flavor | | | |
| Water | QS | QS | QS |

*Actives used individually or in combinations shown in example formulations.

Of the pharmaceutically active compounds described above, those which are particularly preferred are set forth below along with preferred ranges for their inclusion into the claimed liquid excipient base.

Guaifenesin may be present in amounts of from about 25 to about 250 milligrams per 5 ml of the excipient base. Preferably, guaifenesin is present in amounts of from about 50 to about 150 milligrams per 5 ml of the excipient base. Most preferably, guaifenesin is present in amounts of about 100 milligrams per 5 ml of the excipient base.

Dextromethorphan may be present in amounts of from about 0.5 to about 25 milligrams per 5 ml of the excipient base. Preferably, dextromethorphan is present in amounts of from about 5 to about 15 milligrams per 5 ml of the excipient base. Most preferably, dextromethorphan is present in amounts of about5 milligrams per 5 ml of the excipient base.

Brompheniramine/chlorphenimarine may be present in amounts of from about 0.5 to about 2.5 milligrams per 5 ml of the excipient base. Preferably, brompheniramine/chlorphenimarine is present in amounts of from about 1 to about 2 milligrams per 5 ml of the excipient base. Most preferably, brompheniramine/chlorphenimarine is present in amounts of about 1 milligram per 5 ml of the excipient base.

Pseudoephedrine may be present in amounts of from about 5 to about 30 milligrams per 5 ml of the excipient base. Preferably, pseudoephedrine is present in amounts of from about 15 to about 20 milligrams per 5 ml of the excipient base. Most preferably, pseudoephedrine is present in amounts of about 15 milligrams per 5 ml of the excipient base.

Acetominophen may be present in amounts of from about 50 to about 350 milligrams per 5 ml of the excipient base. Preferably, acetaminophen is present in amounts of from about 100 to about 200 milligrams per 5 ml of the excipient base. Most preferably, acetaminophen is present in amounts of about 160 milligrams per 5 ml of the excipient base.

Other components of excipient bases useful in the practice of the present invention are those known to the art. These include humectants such as polyethylene glycol, glycerin and propylene glycol; preservatives such sodium benzoate and paraben; sweeteners such as sodium saccharin, acesulfame potassium, aspartame, corn syrup and sorbitol solutions; menthol; and various flavoring and coloring agents.

### Sensory Evaluation

A sensory study can be conducted to evaluate product taste and flavor preference. Statistical differences will indicate flavor preference. For example, flavor preference can be evaluated in a taste study by subjects who met the inclusion and exclusion criteria (the parameters for these criteria depend on the nature of the product under consideration and are determined in accordance with principals well known to those of skill in the art). Various dependent and response variables can be measured in order to determine flavor preferences. Exemplary response variables (i.e., sensory attributes) are listed in Tables 2 and 3.

TABLE 2
Primary Response Variables*

| Attribute | Rating Scale |
|---|---|
| Flavor | 1 = Dislike Extremely, . . . , 9 = Like Extremely |
| Flavor Intensity | 1 = Not Strong, . . . , 9 = Extremely Strong |
| Sweetness | 1 = Not Sweet, . . . 9 = Extremely Sweet |
| Bitterness | 1 = Not Bitter, . . . 9 = Extremely Bitter |
| Aftertaste | 1 = No Aftertaste, . . . 9 = Extreme Aftertaste |
| Overall Flavor | 1 = Most Preferred, . . . 5 = Least Preferred |
| Preference | |

*All primary response variables are treated as a continuous random variable.

TABLE 3
Secondary Response Variables

| | | Response |
|---|---|---|
| Attribute | Rating Scale | Type |
| Flavor Intensity | 1 = Weaker, 2 = Stronger, 3 = No | Nominal |
| Direction | Change | |
| Sweetness Level | 1 = Less Sweet, 2 = More Sweet, | Nominal |
| Direction | 3 = No Change | |
| Tastes Great | 1 = Agree Completely, . . . , | Continuous |
| | 5 = Disagree Completely | |
| Purchase Intent | 1 = Definitely would | Continuous |
| | buy/recommend, . . . , | |
| | 5 = Definitely would NOT | |
| | buy/recommend | |

Preferably, subjects are randomly assorted in different sequence groups, with each sequence group tasting the samples in different order. Depending on the number of samples to be tested, it may be desirable to use an incomplete block design, in which each subject tastes a fraction of the total number of samples. Such a design is possible when there are a sufficient number of tasters to provide statistically meaningful data.

Sample preparation, presentation to subjects, and evaluation by subjects are conducted according to a protocol prepared in advance of the study. Preferably, before each sample evaluation, each subject cleanses his or her palate with water, soda crackers or some other neutral flavored food or preferably a combination of the two.

Data from the subject reports are entered on an appropriate spread sheet and analyzed statistically. Randomization and, if in place, compliance with double blind provisions, is verified.

Statistical methodology is well known to those of skill in the art. For analysis of continuous variable responses, a univariate analysis of variance crossover model as shown below can be used:

Yᵢⱼₖ=subjectᵢ+sample orderⱼ+prototypeₖ+∈ᵢⱼₖ

where Yᵢⱼₖ is the response of the i^{·th} subject evaluating the j^{·th} sample consisting of the k^{th} prototype; and ∈ᵢⱼₖ is the random error associated with Yᵢⱼₖ.

If an overall difference among the prototypes is detected at the alpha=0.05 significance level, Dunnett's test can be performed on the prototype least squares means. Dunnett's procedure (2-tailed) can show which prototypes differ from the control prototype at the 0.05 significance level.

For analysis of categorical response variables, the statistician may compute the distribution of responses for each prototype. If the effort to implement appropriate statistical methodology would require too much time, inferential tests need not be performed.

For each sensory attribute, the summary measures (least squares means or percentages) from the statistical analysis and the prototype rank order (1:best, . . . , 7 to worst) are listed. The rankings give relative information for comparing the prototypes, while the summary measures give individual prototype information. For example, consider “flavor”. A 9-point attribute scale was characterized as 1 (“dislike extremely”), 5 (“neither like nor dislike”), and 9 (“like extremely”).

For “flavor intensity direction” a point scale is defined as 1=weaker, 2=stronger, or 3=no change. Prototype with the highest percentage of “no change” responses (e.g., 80.00) ranks first (No. 1) (most preferred). In contrast, a prototype with the lowest percentage of “no change” responses has the lowest rank (least preferred).

The other attributes have similar interpretations.

The results of this study establish that compositions containing sucralose at an acid pH have better taste masking qualities.

### EXAMPLES

The invention will now be described with respect to the following specific examples, which are provided by way of illustration and not limitation.

### Example 1

### Flu Syrup

A formulation of the invention set forth below in Table 4 was prepared in accordance with the following procedure. The example illustrates a formulation containing 100 milligrams of sucralose per 100 ml of formulation with a pH of 4.3.

TABLE 4

| Ingredients | % (w/v) | g/1 L | |
|---|---|---|---|
| Acetaminophen | 3.20% | 32 | g |
| Pseudoephedrine Hydrochloride | 0.30% | 3 | g |
| Dextromethorphan Hydrobromide | 0.10% | 1 | g |
| Chlorpheniramine Maleate | 0.02% | .2 | g |
| Polyethylene Glycol 1450 | 20.00% | 200 | g |
| Glycerin | 6.00% | 60 | g |
| High Fructose Corn Syrup 55% | 45.00% | 450 | g |
| Citric Acid Anhydrous | 0.40% | 4 | g |
| Sorbitol, 70% | 5.00% | 50 | g |
| Sodium Citrate | 0.10% | 1 | g |
| Sodium Benzoate | 0.30% | 3 | g |
| Yellow #6 | 0.01% | .1 | g |
| Red #33 | 0.0036% | 36 | mg |
| Sucralose | 0.10% | 1 | g |
| Mixed Berry | 0.10% | 1 | g |
| Water | QS to 100% | | |

| | |
|---|---|
| Final pH | 4.3 |
| Specification/Range pH | 4.0-4.7 |

In a 1500 ml main beaker, PEG 1450 was dissolved in 300 ml of water. Glycerin was added and mixed until the solution was homogenous. Acetaminophen was then dissolved in the solution. In a separate 50 ml beaker, pseudoephedrine, dextromethorphan and chlorpheniramine were dissolved in 15 ml of water. The contents of the 50 ml beaker were then added to the main beaker. Sucralose, sodium benzoate and sodium citrate were then dissolved in 15 ml of water in a separate 50 ml beaker. The contents were mixed until completely dissolved and then added to the main beaker. In a separate 50 ml beaker, citric acid and color were dissolved in 15 ml of water. These contents were then added to the main beaker and mixed well. Sorbitol and high fructose corn syrup were added to the main beaker and mixed until the solution was homogenous. Flavor was added to the main beaker and the volume was adjusted to 1000 ml by adding water and mixing well.

The resulting formulation had a pH of 4.3. In an informal, uncontrolled tasting in the laboratory, the formulation tasted better than a similar formulation at greater pH.

### Example 2

### Sore Throat and Congestion Syrup

A formulation as set forth in Table 5 was prepared.

TABLE 5

| Ingredients | % w/v | g/1 L | |
|---|---|---|---|
| Acetaminophen | 3.20% | 32 | g |
| Pseudoephedrine Hydrochloride | 0.30% | 3 | g |
| Brompheniramine Maleate | 0.02% | .2 | g |
| Polyethylene Glycol 1450 | 20.00% | 200 | g |
| Glycerin | 6.00% | 60 | g |
| High Fructose Corn Syrup 55% | 45.00% | 450 | g |
| Citric Acid Anhydrous | 0.40% | 4 | g |
| Sorbitol, 70% | 5.00% | 50 | g |
| Sodium Citrate | 0.10% | 1 | g |
| Sodium Benzoate | 0.30% | 3 | g |
| Yellow #6 | 0.01% | .1 | g |
| Red #33 | 0.0036% | 36 | mg |
| Bubblegum | 0.10% | 1 | g |
| Sucralose | 0.175% | 1.75 | g |
| Water | QS to 100% | | |

| | |
|---|---|
| Final pH | 4.3 |
| Specification/Range pH | 4.0-5.0 |

The procedure for this formulation was essentially the same as in the previous example except that dextromethorphan was not added and brompheniramine was used instead of chlorpheniramine.

The resulting formulation had a pH of 4.3. In an informal, uncontrolled tasting in the laboratory, the formulation tasted better than a similar formulation at higher pH.

### Example 3

### Cough and Cold Syrup

A formulation as set forth below in Table 6 was prepared.

TABLE 6

| Ingredients | % (w/v) | g/1 L |
|---|---|---|
| Guaifenesin | 2.00% | 20 g |
| Pseudoephedrine Hydrochloride | 0.60% | 6 g |
| Dextromethorphan Hydrobromide | 0.20% | 2 g |
| Polyethylene Glycol 1450 | 20.00% | 200 g |
| Glycerin | 6.00% | 60 g |
| High Fructose Corn Syrup 55% | 45.00% | 450 g |
| Citric Acid Anhydrous | 1.60% | 16 g |
| Sorbitol, 70% | 5.00% | 50 g |
| Sodium Citrate | 0.10% | 1 g |
| Sodium Benzoate | 0.30% | 3 g |
| Red #40 | 0.05% | .5 g |
| Golden Punch Flavor | 0.10% | 1 g |
| Sucralose | 0.20% | 2 g |

| | |
|---|---|
| Water | QS to 100% |
| Final pH | 3.1 |
| Specification/Range pH | 2.6-3.6 |

The procedure for this formulation was essentially the same as in Example 1 except that chlorpheniramine was not added and guaifenesin was used instead of acetaminophen. Different flavors and colors were also used.

The resulting formulation had a pH of 3.1. In an informal, uncontrolled tasting in the laboratory, the formulation tasted better than a similar formulation at higher pH.

### Example 4

### Cough Syrup

A formulation as set forth below in Table 7 was prepared as described.

TABLE 7

| Ingredients | % (w/v) | g/1 L |
|---|---|---|
| Guaifenesin | 2.00% | 20 g |
| Dextromethorphan Hydrobromide | 0.20% | 2 g |
| Polyethylene Glycol 1450 | 20.00% | 200 g |
| Glycerin | 6.00% | 60 g |
| High Fructose Corn Syrup 55% | 45.00% | 450 g |
| Citric Acid Anhydrous | 1.60% | 16 g |
| Sorbitol, 70% | 5.00% | 50 g |
| Sodium Citrate | 0.10% | 1 g |
| Sodium Benzoate | 0.30% | 3 g |
| Red #40 | 0.05% | .5 g |
| Golden Punch Flavor | 0.10% | 1 g |
| Sucralose | 0.20% | 2 g |

| | |
|---|---|
| Water | QS to 100% |
| Final pH | 3.2 |
| Specification/Range pH | 2.7-3.7 |

The procedure for this formulation was essentially the same as in the previous example except that pseudoephedrine was not added.

The resulting formulation had a pH of 3.2. In an informal, uncontrolled tasting in the laboratory, the formulation tasted better than a similar formulation at higher pH.

### Example 5

### Cough and Nasal Decongestant Syrups

A formulation as set forth below in Table 8 was prepared as described.

TABLE 8

| Ingredients | % (w/v) | g/1 L |
|---|---|---|
| Guaifenesin | 2.00% | 20 g |
| Pseudoephedrine Hydrochloride | 0.60% | 6 g |
| Polyethylene Glycol 1450 | 20.00% | 200 g |
| Glycerin | 6.00% | 60 g |
| High Fructose Corn Syrup 55% | 45.00% | 450 g |
| Citric Acid Anhydrous | 1.60% | 160 g |
| Sorbitol, 70% | 5.00% | 50 g |
| Sodium Citrate | 0.10% | 1 g |
| Sodium Benzoate | 0.30% | 3 g |
| Red #40 | 0.05% | .5 g |
| Golden Punch Flavor | 0.10% | 1 g |
| Sucralose | 0.20% | 2 g |

| | |
|---|---|
| Water | QS to 100% |
| Final pH | 3.1 |
| Specification/Range pH | 2.6-3.6 |

The procedure for this formulation was essentially the same as in Example 3 except that dextromethorphan was not added.

The resulting formulation had a pH of 3.1. In an informal, uncontrolled tasting in the laboratory, the formulation tasted better than a similar formulation at higher pH.

### Example 6

### Cough Syrup

A formulation as set forth below in Table 9 was prepared as described.

TABLE 9

| Ingredients | % (w/v) | g/1 L |
|---|---|---|
| Guaifenesin | 2.00% | 20 g |
| Polyethylene Glycol 1450 | 20.00% | 200 g |
| Glycerin | 6.00% | 60 g |
| High Fructose Corn Syrup 55% | 45.00% | 450 g |
| Citric Acid Anhydrous | 1.60% | 16 g |
| Sorbitol, 70% | 5.00% | 50 g |
| Sodium Citrate | 0.10% | 1 g |
| Sodium Benzoate | 0.30% | 3 g |
| Red #40 | 0.05% | .5 g |
| Golden Punch Flavor | 0.10% | 1 g |
| Sucralose | 0.20% | 2 g |

| | |
|---|---|
| Water | QS to 100% |
| Final pH | 2.8 |
| Specification/Range pH | 2.6-3.6 |

The procedure for this formulation was essentially the same as in the previous example except that pseudoephedrine was not added.

The resulting formulation had a pH of 2.8. In an informal, uncontrolled tasting in the laboratory, the formulation tasted better than a similar formulation at higher pH.

### Example 7

### Sensory Preference Analysis

For a specific cough and cold syrup (Robitussin® CF), a sensory study was conducted to evaluate five potential product flavors, four of which were formulated with sucralose. Numerical trends and statistical differences indicate that the golden punch flavor containing sucralose was preferred over the other flavors (citrus passion, juicy apple, grapefruit, and lemon-lime) and the marketed syrup (cherry) phenylpropanolamine replacement product. While this sensory test evaluated flavor preference, it involved comparing a formulation lacking sucralose with numerous formulations containing sucralose, which were generally preferred.

### Primary Research Question(s)

1. How do untrained subjects rate the prototypes with respect to the flavor characteristics?

2. What is the prototype rank order of preference, relative to marketed Robitussin CF Syrup phenylpropanolamine replacement product?

### Primary Dependent or Response Variable(s)

The study response variables (i.e., sensory attributes) are listed in Tables 10 and 11.

TABLE 10
Primary Response Variables*

| Attribute | Rating Scale |
|---|---|
| Flavor | 1 = Dislike Extremely, . . . , 9 = Like Extremely |
| Flavor Intensity | 1 = Not Strong, . . . , 9 = Extremely Strong |
| Sweetness | 1 = Not Sweet, . . . , 9 = Extremely Sweet |
| Bitterness | 1 = Not Bitter, . . . , 9 = Extremely Bitter |
| Aftertaste | 1 = No Aftertaste, . . . , 9 = Extreme Aftertaste |
| Overall Flavor | 1 = Most Preferred, . . . 5 = Least Preferred |
| Preference | |

*All primary response variables are treated as a continuous random variable.

TABLE 11
Secondary Response Variables

| | | Response |
|---|---|---|
| Attribute | Rating Scale | Type |
| Flavor Intensity | 1 = Weaker, 2 = Stronger, 3 = No | Nominal |
| Direction | Change | |
| Sweetness Level | 1 = Less Sweet, 2 = More Sweet, | Nominal |
| Direction | 3 = No Change | |
| Purchase Intent | 1 = Definitely would | Continuous |
| | buy/recommend, . . . , | |
| | 5 = Definitely would NOT | |
| | buy/recommend | |

### Study Design and Explanatory Variables

The study design was an incomplete block design of 6 total samples, 4 samples evaluated by each subject. The design was balanced for prototype effects. A randomization schedule for up to 60 subjects was generated before the study.

The primary explanatory variables are:

subject

prototype

sample order

### Study Execution

According to the written protocol, the study was conducted in the sensory laboratory. Only volunteers who met the inclusion and exclusion criteria in the consent form were allowed to participate. Subjects chose to participate in one of two sessions (a.m. or p.m.).

The sample preparation, presentation (to subjects), and evaluation (by subjects) were conducted according to the protocol. Before each sample evaluation, subjects cleansed their palate with water and/or soda crackers.

### Data Entry and Management

The data from the forms were entered into MS Excel for later statistical analysis. The statistician inspected the worksheet, and resultant SAS dataset for consistency. The randomization schedule was also verified. The data were screened for out-of-range values and corrected for all observed errors. In addition, the statistician wrote the SAS code to build data sets/perform analyses.

### Statistical Methodology

Analysis of Continuous Response Variables. For each continuous response variable, the analysis used the following univariate analysis of variance crossover model:

Yᵢⱼₖ=subjectᵢ+sample orderⱼ+prototypeₖ+∈ᵢⱼₖ

where Yᵢⱼₖ is the response of the i^{·th} subject evaluating the j^{·th} sample consisting of the jprototype; and ∈ᵢⱼₖ is the random error associated with Yᵢⱼₖ.

If an overall difference among the prototypes was detected at the alpha=0.05 significance level, Dunnett's test was performed on the prototype least squares means. Dunnett's procedure (2-tailed) showed which prototypes were different from the control prototype at the 0.05 significance level.

Analysis of Categorical Response Variables. For each categorical response variable, the statistician computed the distribution of responses for each prototype. Because the effort to implement appropriate statistical methodology would require too much time, inferential tests were not performed.

Statistical Software. Descriptive and inferential analyses were conducted on the SAS for Windows System 8.01 (1999-2000).

### Descriptive and Inferential Results

Number of Subjects. Twenty-eight subjects entered and completed the study.

Comparison of Prototypes. For each sensory attribute, the summary measures (least squares means or percentages) from the statistical analysis and the prototype rank order (1:best, . . . , 6:worst) are listed in Table 12. The rankings give relative information for comparing the prototypes, while the summary measures give individual prototype information.

For example, consider “sweetness” in Table 12. The 9-point attribute scale was characterized as 1 (“dislike extremely”), 5 (“neither like nor dislike”) and 9 (“like extremely”). Prototype 090 had the highest mean liking score (4.96) and a rank of 1 (most preferred). In contrast, control prototype 268 had the lowest mean score (3.20) and a rank of 6 (least preferred). While all prototypes had higher mean sweetness scores than the control, marketed Robitussin CF phenylpropanolamine replacement product (268), prototypes 090 and 423 had statistically significant better sweetness scores.

Similarly, the control prototype 268 ranked highest for “bitterness” (mean scores=5.11) compared to all other prototypes containing sucralose.

Consider “flavor” in Table 12. Prototype 090 had the highest mean liking score (5.98) and a rank of 1 (most preferred). In contrast, prototype 336 had the lowest mean score (3.19) and a rank of 6 (least preferred). Only prototype 090 had a statistically significantly better flavor than the marketed Robitussin CF phenylpropanolamine replacement product (268).

For “flavor intensity direction” in Table 12, the 3-point scale was defined as 1=weaker, 2=stronger, or 3=no change. Table 13 lists only the percentage of “no change” responses. Prototype 925 had the highest percentage of “no change” responses (66.67) and a rank of 1 (most preferred). In contrast, prototype 336 had the lowest percentage of “no change” responses (26.32) and a rank of 6 (least preferred).

The other attributes have similar interpretations.

Numeric trends and statistical differences indicate that prototype 090 (golden punch) was preferred over the other prototypes. Note, however, that since in-house volunteers evaluated the prototypes in a controlled environment, the mean scores do not necessarily show actual consumer product preference.

TABLE 12
Summary of Statistical Results by Sensory Attribute

| | Summary Measure | | | | | | Prototype Ranking | | | | | |
|---|---|---|---|---|---|---|---|---|---|---|---|---|
| | Prototype | | | | | | Prototype | | | | | |
| Sensory Attribute | 90 | 268 | 336 | 423 | 839 | 925 | 90 | 268 | 336 | 423 | 839 | 925 |
| Flavor (a) | 5.98* | 4.41 | 3.19 | 4.84 | 4.81 | 4.11 | 1.00 | 4.00 | 6.00 | 2.00 | 3.00 | 5.00 |
| Flavor Intensity (a) | 6.21 | 5.01 | 5.41 | 6.38* | 4.67 | 5.62 | — | — | — | — | — | — |
| Flavor Intensity Direction (b) | 52.63 | 33.33 | 26.32 | 47.37 | 52.63 | 66.67 | 2.50 | 5.00 | 6.00 | 4.00 | 2.50 | 1.00 |
| Sweetness (a) | 4.96* | 3.20 | 3.79 | 4.58* | 4.09 | 3.87 | — | — | — | — | — | — |
| Sweetness Direction (b) | 47.37 | 38.89 | 36.84 | 36.84 | 52.63 | 44.44 | 2.00 | 4.00 | 5.50 | 5.50 | 1.00 | 3.00 |
| Bitterness (a) | 3.78 | 5.11 | 5.07 | 4.43 | 4.47 | 4.70 | 1.00 | 6.00 | 5.00 | 2.00 | 3.00 | 4.00 |
| Aftertaste (a) | 3.59* | 5.41 | 5.80 | 4.76 | 5.04 | 5.18 | 1.00 | 5.00 | 6.00 | 2.00 | 3.00 | 4.00 |
| Purchase Intent (c) | 2.64 | 3.38 | 3.91 | 3.11 | 2.97 | 3.34 | — | — | — | — | — | — |
| Flavor Preference (d) | 1.71* | 2.93 | 3.17 | 1.90 | 2.40 | 2.94 | 1.00 | 4.00 | 6.00 | 2.00 | 3.00 | 5.00 |
| Average | — | — | — | — | — | — | 1.42 | 4.67 | 5.75 | 2.92 | 2.58 | 3.67 |

Summary Measures:
(a): least squares means from ANOVA: (1 = low level, 9 = high level)
(b): percentages of “No change” from the contingency table
(c): least squares means from ANOVA: (1 = would buy, 5 = would not buy)
(d): least squares means from ANOVA: (low score = most preferred, high score = least preferred)
*statiscially “different from” from control prototype 268 at the 0.05 level of significance based on Dunnetts test

### STUDY REFERENCES

Meilgaard, M., Civille, G. V., and Carr, B. T. (1991), Sensory Evaluation Techniques, 2^{nd} Ed., Boca Raton, Fla., CRC Press.

Neter, J., Wasserman, W., and Kutner, M. H. (1990), Applied Linear Statistical Models, Homewood, Ill.: Richard D. Irwin, Inc.

SAS Institute Inc., SAS OnlineDoc®, Version 8, Cary, N.C.: SAS Institute Inc., 1999.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1-30. (canceled)

31. A pharmaceutical composition comprising
(i) a liquid excipient base comprising about 50 mg to about 300 mg of sucralose per 100 mL and having a pH of 2.0 to 5.0;
(ii) about 10 mg to about 50 mg chlorpheniramine per 100 mL;
(iii) a decongestant selected from pseudoephedrine HCl, phenylpropanolamine and phenylephrine; and
(iv) ibuprofen, wherein said decongestant and said ibuprofen are dissolved or suspended in the liquid excipient base.

32. The composition of claim 1 comprising about 100 to about 200 mg of sucralose.

33. The composition of claim 2 comprising about 200 mg of sucralose.

34. The composition of claim 1, wherein the pH is the range of 2.5 to 5.0.

35. A pharmaceutical composition according to claim 1, wherein said decongestant comprises about 0.1-0.6% pseudoephedrine hydrochloride.

36. The pharmaceutical composition of claim 35, further comprising 1.0-5.0% ibuprofen dissolved or suspended in said liquid excipient base.

37. The pharmaceutical composition of claim 35, further comprising 0.5-5.0% of guaifenesin.

38. The pharmaceutical composition of claim 35, further comprising 0.01-0.5% dextromethorphan.

39. The composition of claim 35 comprising about 100 to about 200 mg of sucralose.

40. The composition of claim 39 comprising about 200 mg of sucralose.

41. The composition of claim 34 wherein the pH is in the range of 2.5 to 5.0.

42. The composition of claim 41, wherein the pH is in the range of 2.0 to 3.7.
